(19) United States
(12) Patent Application Publication
(10) Pub. No.: US 2022/0074915 A1
(43) Pub. Date: Mar. 10, 2022

(54) DEVICE AND METHOD FOR PREPARING NATURAL GAS HYDRATE UNDER CONTROLLED TEMPERATURE AND PRESSURE

(71) Applicant: INSTITUTE OF GEOLOGY AND GEOPHYSICS, CHINESE ACADEMY OF SCIENCES, Beijing (CN)

(72) Inventors: Jianming He, Beijing (CN); Yanfang Wu, Beijing (CN); Jian Huo, Beijing (CN); Zhaobin Zhang, Beijing (CN); Shouding Li, Beijing (CN); Xiao Li, Beijing (CN)

(21) Appl. No.: 17/141,086

(22) Filed: Jan. 4, 2021

(30) Foreign Application Priority Data
Sep. 9, 2020 (CN) 202010938958.0

(51) Int. Cl. G01N 33/24

(52) U.S. Cl. CPC G01N 33/241

(57) ABSTRACT

The present disclosure provides a device and a method for preparing natural gas hydrate under controlled temperature and pressure, which relates to the technical field of rock and soil mechanics test technology and equipment. The device includes a three-axis cylinder outer tube, a temperature adjustment system, a pressure loading system, a gas supply system and a recovery system. The three-axis cylinder outer tube is provided with an accommodating cavity throughout its middle, and the accommodating cavity is provided with an end cover of the three-axis cylinder outer tube at each end thereof. The temperature adjustment system is used to adjust the temperature of the three-axis cylinder outer tube. The pressure loading system is used to apply pressure load to the accommodating cavity. The gas supply system is used to supply a high-pressure gas into the accommodating cavity. The recovery system is used to recover the moisture and gas in the accommodating cavity.

### CROSS-REFERENCE TO RELATED APPLICATION

The present application claims the priority to Chinese Patent Application No. 202010938958.0 to He et al., filed Sep. 9, 2020, and entitled “Device and Method for Preparing Natural Gas Hydrate under Controlled Temperature and Pressure”, the disclosure of which is incorporated herein by reference in its entirety.

### TECHNICAL FIELD

The present disclosure relates to the technical field of rock and soil mechanics test technology and equipment, and in particular to a device and a method for preparing natural gas hydrate under controlled temperature and pressure.

### BACKGROUND

Natural gas hydrate, which is an ice-like crystalline substance formed by natural gas and water under a high pressure and a low temperature, is a new type of clean and pollution-free energy with a high combustion value, and is widely distributed in deep sea sediments or permafrost in land. The natural gas hydrate mainly presents in marine sediments or frozen soil particles in the form of pore filling, and the particles of marine sediments or frozen soil are composed of sand, silt and clay. Once the temperature increases or the pressure decreases, methane gas will escape, and solid hydrate will tend to decompose, resulting in that it is difficult to obtain natural gas hydrate in situ. To study natural gas hydrate, it is necessary to simulate the actual environmental conditions in a laboratory to prepare natural gas hydrate and store it under low temperature, so as to be used in subsequent laboratory tests on natural gas hydrate.

### SUMMARY

In order to solve the above technical problems, the present disclosure provides a device and a method for preparing natural gas hydrate under controlled temperature and pressure, so as to simulate the actual environmental conditions in a laboratory to prepare natural gas hydrate and store it.

In order to achieve the above objective, the present disclosure provides the following technical solutions:

The present disclosure provides a device for preparing natural gas hydrate under controlled temperature and pressure, comprising a three-axis cylinder outer tube, a temperature adjustment system, a pressure loading system, a gas supply system and a recovery system; the three-axis cylinder outer tube is provided with an accommodating cavity throughout its middle, and the accommodating cavity is provided with an end cover of the three-axis cylinder outer tube at each end thereof; the temperature adjustment system is used to adjust the temperature of the three-axis cylinder outer tube; the pressure loading system is used to apply pressure load to the accommodating cavity; the gas supply system is used to supply a high-pressure gas into the accommodating cavity; the recovery system is used to recover the moisture and gas in the accommodating cavity.

In some embodiments, the temperature adjustment system comprises a cold bath heat exchange container, a liquid circulation pump and a temperature-controlled cold bath device; the cold bath heat exchange container is arranged outside the three-axis cylinder outer tube, and the liquid circulation pump, the temperature-controlled cold bath device and the cold bath heat exchange container are communicated through pipelines in sequence.

In some embodiments, the pressure loading system comprises an axial pressure loading servo pump and a confining pressure loading servo pump, and both the axial pressure loading servo pump and the confining pressure loading servo pump communicate with the accommodating cavity.

In some embodiments, the gas supply system comprises a gas source and a gas booster pump; the gas booster pump communicates with the gas source at the gas inlet of the pump, and communicates with the accommodating cavity at the gas outlet of the pump.

In some embodiments, the recovery system comprises a back pressure valve, a gas-liquid separator and a methane recovery tank that are connected in sequence; the back pressure valve communicates with the accommodating cavity at the gas inlet end of the valve.

The present disclosure provides a method for preparing natural gas hydrate under controlled temperature and pressure with the above device, comprising the following steps:

Step 1: reshaping a sand sample in a laboratory according to the requirements of actual natural gas hydrate for the constituent particles and the particle size thereof; placing a water filter plate and a sample cushion block at each upper end and lower end of the reshaped sand sample, and integrally encapsulating them with a heat shrinkable tube to form a reshaped sample; after that, connecting the encapsulated reshaped sample with a glassware by a transparent connecting tube; adding deionized water to the glassware until the water level is higher than the reshaped sample, and continuously adding deionized water for a certain period of time until the water level is stable and unchanged; encapsulating the saturated reshaped sample with a heat shrinkable tube and then connecting the two ends of the sample to two pressure touch control valves respectively, so as to ensure the water in the saturated sample cannot be discharged;

Step 2: pushing the reshaped sample that encapsulated with the heat shrinkable tube and under a sealed state into a three-axis cylinder outer tube, connecting the air supply system to the pressure touch control valve at the lower end of the reshaped sample, and connecting the recovery system to the pressure touch control valve at the upper end of the encapsulated sample, then placing an end cushion block at each end of the three-axis cylinder outer tube, and finally sealing the three-axis cylinder outer tube with an end cover at each end thereof;

Step 3: connecting an axial pressure loading servo pump to the axial pressure loading connection port of the three-axis cylinder outer tube; connecting a confining pressure loading servo pump to the confining pressure loading connection port of the three-axis cylinder outer tube; tightly sleeving a cold bath heat exchange container, which is filled with cold bath circulating liquid, outside the three-axis cylinder outer tube to exchange heat, so as to control the temperature of the reshaped sample inside the three-axis cylinder outer tube; connecting a liquid circulation pump and a temperature-controlled cold bath device to the cold bath heat exchange container through a cold bath connection inlet pipeline and a cold bath connection loop;

Step 4: controlling the confining pressure loading servo pump and the axial pressure loading servo pump to make the confining pressure and the axial pressure acting on the reshaped sample reach the pressure to be simulated in sequence; at this time, the pressure touch control valves are opened by the axial pressure, so that the methane gas stored in the methane storage tank enters the water-saturated reshaped sample under the action of a gas booster pump through the methane gas inlet pipeline and reaches saturation in water; starting the temperature-controlled cold bath device and the liquid circulation pump, and controlling the temperature of the reshaped sample inside the three-axis cylinder outer tube by the heat exchange through the circulation of the cold bath circulating fluid, so as to realize the control of the temperature and pressure under which the reshaped sample is located; at this time, the methane gas passing through the reshaped sample starts to react with the water under conditions of high-pressure and low-temperature to obtain natural gas hydrate, wherein the remaining methane gas and water enter a gas-liquid separator through a gas-liquid loop and a back pressure valve, and the separated gas enters the methane recovery tank;

Step 5: after the synthesis of the natural gas hydrate is finished, closing the axial pressure loading servo pump and confining pressure loading servo pump; at this time, the pressure touch control valve is closed and the reshaped sample is in a closed pressure-holding state; opening the three-axis cylinder outer tube end covers, connecting the three-axis cylinder outer tube with the outer cavity of a globe-valve, and transferring the reshaped sample into the sphere of the globe-valve, wherein the two end cushion blocks of the three-axis cylinder outer tube enter the two ends of the outer cavity of the globe-valve respectively; after the transferring is completed, rotating the sphere of the globe-valve to completely seal the reshaped sample; placing the globe-valve under low temperature to realize the pressure-holding storage of the natural gas hydrate.

Compared with the prior art, the present disclosure has achieved the following technical effects: the preparation and pressure-holding storage of the natural gas hydrate are realized by simulating geological environment with high-pressure and low-temperature in hydrate reservoirs in a laboratory; a high-saturated natural gas hydrate sample is generated by introducing fluid methane gas into the reshaped saturated sand sample, which solves the problems emerging in the laboratory preparation of natural gas hydrate; the sealed pressure-holding transfer of a sample is realized by the connection of the three-axis cylinder outer tube with a globe-valve, which solves the problem of the change in the property caused by the loss of pressure during the sample transfer; the natural gas hydrate generated is stored in a globe-valve under a sealed and pressure-holding condition, which solves the problems emerging in the storage of the gas hydrate in the laboratory under a sealed and pressure-holding condition.

### BRIEF DESCRIPTION OF THE DRAWINGS

In order to illustrate the embodiments of the present disclosure or the technical solutions in the prior art more clearly, the drawings used in the embodiments will be briefly introduced below. Obviously, the drawings in the following description are only some embodiments of the present disclosure, and other drawings can be obtained according to these drawings for the ordinary skilled in the art without creative labor.

FIG. 1 shows a schematic diagram of water saturation of reshaped samples.

FIG. 2 shows a schematic diagram of the preparation of natural gas hydrate under controlled temperature and pressure.

FIG. 3 shows a schematic diagram of a natural gas hydrate in a globe-valve.

FIG. 4 shows a schematic diagram of the storage of natural gas hydrate under a sealed and pressure-holding condition.

Description of reference numerals: 1: reshaped sample; 2: water filter plate; 3: sample cushion block; 4: transparent connecting pipe; 5: glassware; 6: three-axis cylinder outer tube; 7: end cover of the three-axis cylinder outer tube; 8: end cushion block of the three-axis cylinder outer tube; 9: confining pressure loading connection port; 10: axial pressure loading connection port; 11: pressure touch control valve; 12: confining pressure connecting pipeline; 13: axial pressure connecting pipeline; 14: cold bath connecting loop; 15: axial pressure loading servo pump; 16: confining pressure loading servo pump; 17: liquid circulation pump; 18: temperature-controlled cold bath device; 19: cold bath connection inlet pipeline; 20: cold bath heat exchange container; 21: cold bath circulating liquid; 22: connecting pipeline of axial pressure loading cavity; 23: gas-liquid loop; 24: back pressure valve; 25: outer cavity of the globe-valve; 26: sphere of the globe-valve; 27: methane gas inlet pipeline; 28: gas booster pump; 29: methane storage tank; 30: gas-liquid separator; 31: methane recovery loop; 32: methane recovery tank.

### DETAILED DESCRIPTION

The technical solution in the examples of the present disclosure will be described clearly and completely with reference to the drawings in the examples of the present disclosure. Obviously, the examples as described are only part of the examples of the present disclosure, not all of them. Based on the examples of the present disclosure, all other examples obtained by the ordinary skilled in the art without creative labor should be within the protection scope of the present disclosure.

### Example 1

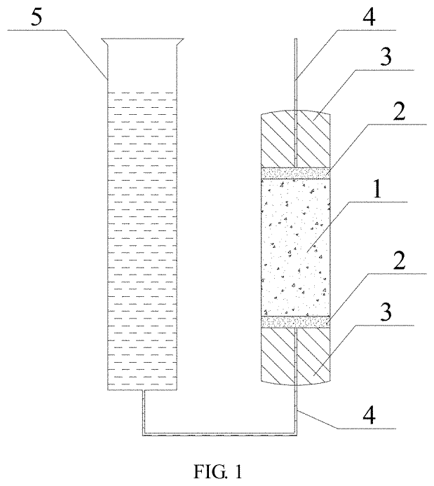

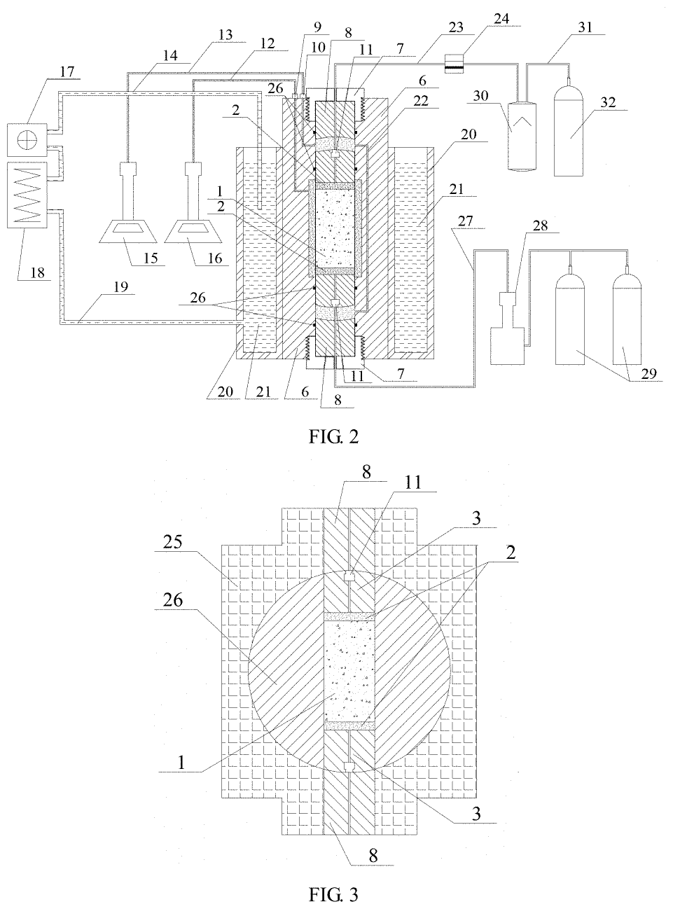

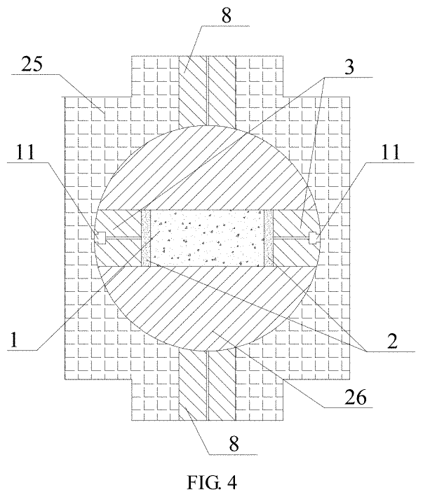

As shown in FIGS. 1 and 2, this example provides a device for preparing natural gas hydrate under controlled temperature and pressure, comprising a three-axis outer tube 6, a temperature adjustment system, a pressure loading system, a gas supply system and a recovery system; the three-axis cylinder outer tube 6 is provided with an accommodating cavity throughout its middle, and the accommodating cavity is provided with an end cover of the three-axis cylinder outer tube 7 at each end thereof; the temperature adjustment system is used to adjust the temperature of the three-cylinder outer tube 6; the pressure loading system is used to apply pressure load to the accommodating cavity; the gas supply system is used to supply a high-pressure gas into the accommodating cavity; the recovery system is used to recover the moisture and gas in the accommodating cavity.

In this specific example, the temperature adjustment system comprises a cold bath heat exchange container 20, a liquid circulation pump 17 and a temperature-controlled cold bath device 18; the cold bath heat exchange container 20 is arranged outside the three-axis cylinder outer tube 6, and the liquid circulation pump 17, the temperature-controlled cold bath device 18 and the cold bath heat exchange container 20 are communicated through pipelines in sequence.

The pressure loading system comprises an axial pressure loading servo pump 15 and a confining pressure loading servo pump 16; both the axial pressure loading servo pump 15 and the confining pressure loading servo pump 16 communicate with the accommodating cavity.

The gas supply system comprises a methane storage tank 29 and a gas booster pump 28; the gas booster pump 28 communicates with the methane storage tank 29 at the gas inlet of the pump 28, and communicates with the accommodating cavity at the gas outlet of the pump 28.

The recovery system comprises a back pressure valve 24, a gas-liquid separator 30 and a methane recovery tank 32 that are connected in sequence; the back pressure valve 24 communicates with the accommodating cavity at the gas inlet end of the valve 24.

The three-axis cylinder outer tube 6 is provided with an axial pressure loading connection port and a confining pressure loading connection port, wherein one end of the axial pressure loading connection port is communicated with the axial pressure loading servo pump 15, the other end thereof is communicated with the upper and lower ends of the reshaped sample 1, and one end of the confining pressure loading connection port is communicated with the confining pressure loading servo pump 16, the other end thereof is communicated with the periphery of the reshaped sample 1.

### Example 2

This example provides a method for preparing natural gas hydrate under controlled temperature and pressure with the device as provided in Example 1, specifically comprising the following steps:

Step 1: a sand sample is reshaped in a laboratory according to the requirements of an actual natural gas hydrate for the constituent particles and particle size thereof; a water filter plate 2 and a sample cushion block 3 are placed at each upper end and lower end of the reshaped sand sample, and they are integrally encapsulated with a heat shrinkable tube to form a reshaped sample 1; after that, the encapsulated reshaped sample 1 is connected with a glassware 5 by a transparent connecting tube 4; deionized water is added to the glassware 5 until the water level is higher than the reshaped sample 1, which could be observed by the transparent connecting tube 4, and deionized water is added continuously for a certain period of time until the water level is stable and unchanged; the saturated reshaped sample is encapsulated with a heat shrinkable tube and then each end of the sample is connected to a pressure touch control valve 11, so as to ensure the water in the saturated sample cannot be discharged.

Step 2: the reshaped sample 1 that encapsulated with the heat shrinkable tube and under a sealed state is pushed into the three-axis cylinder outer tube 6; a methane gas storage tank 29 and a gas booster pump 28 are in sequence connected to a pressure touch control valve 11 at the lower end of the encapsulated reshaped sample through a methane gas inlet pipeline 27; a gas-liquid separator 30 and a back pressure valve 24 are in sequence connected to a pressure touch control valve 11 at the upper end of the encapsulated reshaped sample through a gas-liquid loop 23; a methane recovery tank 32 is connected to the gas-liquid separator 30 through a methane recovery loop 31; after that, an end cushion block of the three-axis cylinder outer tube 8 is placed at each end of the three-axis cylinder outer tube 6; finally, the three-axis cylinder outer tube 6 is sealed with an end cover 7 at each end thereof.

Step 3: an axial pressure loading servo pump 15 is connected to an axial pressure loading connection port 10 through an axial pressure connection pipeline 13; a confining pressure loading servo pump 16 is connected to a confining pressure loading connection port 9; a cold bath heat exchange container 20, which is filled with cold bath circulating liquid 21, is tightly sleeved outside the three-axis cylinder outer tube 6 to exchange heat, so as to control the temperature of the reshaped sample 1 inside the three-axis cylinder outer tube 6; a liquid circulation pump 17 and a temperature-controlled cold bath device 18 are connected to the cold bath heat exchange container 20 through a cold bath connection inlet pipeline 19 and a cold bath connection loop 14.

Step 4: the confining pressure loading servo pump 16 and the axial pressure loading servo pump 15 are controlled to make the confining pressure and the axial pressure acting on the reshaped sample 1 reach the pressure to be simulated; at this time, the pressure touch control valves 11 are opened by the axial pressure, so that the methane gas stored in the methane storage tank 29 enters the water-saturated reshaped sample under the action of the gas booster pump 28 through the methane gas inlet pipeline 27 and reaches saturation in water; the temperature-controlled cold bath device 18 and the liquid circulation pump 17 are started, and the temperature of the reshaped sample 1 inside the three-axis cylinder outer tube 6 is controlled by the heat exchange through the circulation of the bath circulating fluid 21, so as to realize the control of the temperature and pressure under which the reshaped sample 1 is located; at this time, the methane gas passing through the reshaped sample 1 starts to react with the water under conditions of high-pressure and low-temperature to obtain natural gas hydrate, wherein the remaining methane gas and water enter the gas-liquid separator 30 through the gas-liquid loop 23 and the back pressure valve 24, and the separated gas enters the methane recovery tank 32.

Step 5: after the synthesis of the natural gas hydrate is finished, the axial pressure loading servo pump 15 and confining pressure loading servo pump 16 are closed; at this time, the pressure touch control valve 11 is closed and the reshaped sample 1 is in a closed pressure-holding state; the end covers of the three-axis cylinder outer tube 7 are opened, the three-axis cylinder outer tube 6 is connected with the outer cavity of a globe-valve 25, and the reshaped sample 1, water filter plate 2 and sample cushion block 3, which have been encapsulated together by a heat shrinkable tube, are transferred into the sphere of the globe-valve 26 integrally, wherein the end cushion blocks of the three-axis cylinder outer tube 8 enter the two ends of the outer cavity of the globe-valve 25 respectively; after the transferring is completed, the sphere of the globe-valve 26 is rotated to completely seal the reshaped sample 1, water filter plate 2 and sample cushion block 3, which have been encapsulated together by a heat shrinkable tube; the pressure-holding storage of natural gas hydrate could be realized by placing the globe-valve under low temperature.

It should be noted that it is obvious to those skilled in the art that the present disclosure is not limited to the details of the above exemplary examples, and that the present disclosure can be realized in other specific forms without departing from the spirit or basic characteristics of the present disclosure. Therefore, from any point of view, the examples should be regarded as exemplary and non-limiting, and the scope of the disclosure is defined by the appended claims rather than the above description, so that it is intended to include all changes falling within the meaning and scope of the equivalent elements of the claims, and any reference signs in the claims should not be regarded as limiting the claims involved.

In this specification, specific examples are applied to illustrate the principle and embodiments of the present disclosure, and the illustration of the above examples is only used to help understand the method of the present disclosure and its core ideas; At the same time, according to the idea of the present disclosure, there will be some changes in the specific embodiment and application scope for the ordinary technicians in the field. In summary, the contents of this specification should not be understood to limit the present disclosure.

What is claimed is:

1. A device for preparing natural gas hydrate under controlled temperature and pressure, comprising:
a three-axis cylinder outer tube,
a temperature adjustment system,
a pressure loading system, and
a gas supply system and a recovery system;
the three-axis cylinder outer tube is provided with an accommodating cavity throughout its middle, and the accommodating cavity is provided with an end cover of the three-axis cylinder outer tube at each end thereof;
the temperature adjustment system is used to adjust the temperature of the three-axis cylinder outer tube;
the pressure loading system is used to apply pressure load to the accommodating cavity;
the gas supply system is used to supply a high-pressure gas into the accommodating cavity;
the recovery system is used to recover the moisture and gas in the accommodating cavity.

2. The device as claimed in claim 1, wherein the temperature adjustment system comprises
a cold bath heat exchange container, and
a liquid circulation pump and a temperature-controlled cold bath device;
the cold bath heat exchange container is arranged outside the three-axis cylinder outer tube, and the liquid circulation pump, the temperature-controlled cold bath device and the cold bath heat exchange container are communicated through pipelines in sequence.

3. The device as claimed in claim 1, wherein the pressure loading system comprises
an axial pressure loading servo pump and a confining pressure loading servo pump;
both the axial pressure loading servo pump and the confining pressure loading servo pump communicate with the accommodating cavity.

4. The device as claimed in claim 1, wherein the gas supply system comprises a gas source and a gas booster pump; the gas booster pump communicates with the gas source at the gas inlet of the pump, and communicates with the accommodating cavity at the gas outlet of the pump.

5. The device as claimed in claim 1, wherein the recovery system comprises
a back pressure valve, a gas-liquid separator and a methane recovery tank that are connected in sequence;
the back pressure valve communicates with the accommodating cavity at the gas inlet end of the valve.

6. A method for preparing natural gas hydrate under controlled temperature and pressure with the device as claimed in claim 1, comprising the following steps:
step 1: reshaping a sand sample in a laboratory according to the requirements of an actual natural gas hydrate for the constituent particles and particle size thereof, placing a water filter plate and a sample cushion block at each upper end and lower end of the reshaped sand sample, and integrally encapsulating them with a heat shrinkable tube to form a reshaped sample; after that, connecting the encapsulated reshaped sample with a glassware by a transparent connecting tube; adding deionized water to the glassware until the water level is higher than the reshaped sample, and continuously adding deionized water for a certain period of time until the water level is stable and unchanged; encapsulating the saturated reshaped sample with a heat shrinkable tube and connecting the two ends of the sample to two pressure touch control valves respectively, so as to ensure the water in the saturated sample cannot be discharged;
step 2: pushing the reshaped sample that encapsulated with the heat shrinkable tube and under a sealed state into a three-axis cylinder outer tube, connecting the air supply system to the pressure touch control valve at the lower end of the reshaped sample, and connecting the recovery system to the pressure touch control valve at the upper end of the encapsulated sample, then placing a three-axis cylinder end cushion block at each end of the three-axis cylinder outer tube, and finally sealing the three-axis cylinder outer tube with an end cover at each end thereof;
step 3: connecting an axial pressure loading servo pump to the axial pressure loading connection port of the three-axis cylinder outer tube, connecting a confining pressure loading servo pump to the confining pressure loading connection port of the three-axis cylinder outer tube; tightly sleeving a cold bath heat exchange container, which is filled with cold bath circulating liquid, outside the three-axis cylinder outer tube to exchange heat, so as to control the temperature of the reshaped sample inside the three-axis cylinder outer tube; connecting a liquid circulation pump and a temperature-controlled cold bath device to the cold bath heat exchange container through a cold bath connection inlet pipeline and a cold bath connection loop;
step 4: controlling the confining pressure loading servo pump and the axial pressure loading servo pump to make the confining pressure and the axial pressure acting on the reshaped sample reach the pressure to be simulated; at this time, the pressure touch control valves are opened by the axial pressure, so that the methane gas stored in the methane storage tank enters the water-saturated reshaped sample under the action of a gas booster pump through the methane gas inlet pipeline and reaches saturation in water; starting the temperature-controlled cold bath device and the liquid circulation pump, and controlling the temperature of the reshaped sample inside the three-axis cylinder outer tube by the heat exchange through the circulation of the cold bath circulating fluid, so as to realize the control of the temperature and pressure under which the reshaped sample is located; at this time, the methane gas passing through the reshaped sample starts to react with the water under conditions of high-pressure and low-temperature to obtain natural gas hydrate, wherein the remaining methane gas and water enter the gas-liquid separator through a gas-liquid loop and a back pressure valve, and the separated gas enters the methane recovery tank;
step 5: after the synthesis of the natural gas hydrate is finished, closing the axial pressure loading servo pump and confining pressure loading servo pump; at this time, the pressure touch control valve is closed and the reshaped sample is in a closed pressure-holding state; opening the end covers of the three-axis cylinder outer tube, connecting the three-axis cylinder outer tube with the outer cavity of a globe-valve, and transferring the reshaped sample into the sphere of the globe-valve, wherein the two end cushion blocks of the three-axis cylinder outer tube enter the two ends of the outer cavity of the globe-valve respectively; after the transferring is completed, rotating the sphere of the globe-valve to completely seal the reshaped sample, and placing the globe-valve under low temperature to realize the pressure-holding storage of the natural gas hydrate.
